(19) United States
(12) Patent Application Publication
(10) Pub. No.: US 2006/0052728 A1
(43) Pub. Date: Mar. 9, 2006

(54) Dynamic oscillating gait-training system

(76) Inventors: D. Casey Kerrigan, Charlottesville, VA (US); Patrick O. Riley, Charlottesville, VA (US); Ugo Della Croce, Boston, MA (US); Paul E. Allaire, Charlottesville, VA (US); Jun-Ho Lee, Amyang-City (KR)

Correspondence Address: UNIVERSITY OF VIRGINIA PATENT FOUNDATION, 250 WEST MAIN STREET, SUITE 300, CHARLOTTESVILLE, VA, 22902, US

(21) Appl. No.: 11/194,743

(22) Filed: Aug. 1, 2005

Related U.S. Application Data
(60) Provisional application No. 60/592,679, filed on Jul. 30, 2004.

(51) Int. Cl. A61B 5/11; A61B 5/103; A63B 22/02; G01M 19/00

(52) U.S. Cl. 600/595; 482/54; 73/172; 128/905

(57) ABSTRACT

Partial body-weight support systems and methods for human gait training are disclosed. The system may include a motor and a cord coupled to the motor, the cord and the motor providing at least partial body-weight support to a user during human gait training. The system may also include a controller that actively adjusts the operation of the motor based upon measured gait parameters. The method may include coupling a human to a motor and monitoring the gait of the human while the human is walking to produce a signal indicative of the gait of the human. The method may also include varying the operation of the motor based on the signal so as to assist the human in attaining a predetermined gait pattern.

### CROSS REFERENCE TO RELATED APPLICATIONS

The present application claims priority from U.S. Provisional Application No. 60/592,679, filed Jul. 30, 2004, which is incorporated herein by reference.

### TECHNICAL FIELD

The present invention relates to systems and methods for human gait training. In particular, the present invention relates to systems and methods for training a human's gait by varying the operation of a motor providing partial body weight support based on measured gait parameters.

### BACKGROUND ART

Millions of people in the United States have difficulty walking as a result of various neurological injuries including stroke, spinal cord injury, cerebral palsy, and traumatic brain injury, as well as musculoskeletal injuries including fractures, joint replacements, and ligament and tendon injuries. For these people, perhaps the most promising rehabilitation strategy for the recovery of walking (gait) is partial body-weight support (PBWS) gait training. Studies support the concept that specific sensory input signals enhance the reflex function of the intrinsic control, i.e., the central pattern generator for gait, and facilitate retraining motor function. Preliminary studies in humans with neurological injury have shown that PBWS gait-training protocols as short as 3 to 6 weeks can improve walking function as significantly as conventional rehabilitation, which may last up to 6 years.

Beyond patient populations with neurological and musculoskeletal injuries are a far larger number of persons who wish to obtain the fitness benefits of walking or running, but who need to or wish to avoid undue joint and muscle loading. With proper supervision, PBWS can provide fitness benefits in either a rehabilitation or health club setting.

Currently existing PBWS gait training devices are all passive devices where the applied body weight force is not under active control. They can be categorized as 1) passive systems with a cable/harness attached to a fixed bar or structure above the body that provides direct suspension support with the body center of mass (CoM) kept nearly vertically fixed in space by a relatively rigid harness attached to the upper body, 2) force-offset systems that utilize counter-weights to apply a constant vertically upward force to the subject regardless of the person's displacement (in which case the CoM inertial/acceleration forces are amplified rather than reduced), and 3) elastic tensioned systems with spring mechanisms or balloon devices wherein the upward force increases in proportion to downward displacement.

### SUMMARY OF THE INVENTION

The systems and methods of the present invention may provide, among other things, partial body-weight support, the amount of which may vary through the gait cycle to allow for a natural gait pattern such as a natural center of mass oscillation. The system may utilize a feedback controller that actively adjusts forces to or displacements of the subject's body based on measured gait parameters, wherein the magnitude of the forces to or displacements of the body varies throughout the subject's gait cycle. The system may be used for training or diagnosis of a subject.

More particularly, one embodiment of the present invention is directed to a dynamic oscillating gait system. This embodiment may control body movement and provide mechanical and sensory inputs to produce a natural gait pattern, maximally using a person's residual and developing function, and may require minimal rehabilitation and training manpower. This embodiment advantageously overcomes some or all of the problems with the systems described herein. Namely, embodiments of the present invention may reduce the number of workers needed to assist the patient and may also aid in activating the hip, knee, and ankle flexor/extensors in a way to effectively rehabilitate gait or improve fitness.

In one embodiment, the present invention is directed to partial body-weight support system for human gait training. The system of this embodiment may include a motor. In this embodiment, the motor may be any type of motor but, in particular, the motor may be a linear motor. The system of this embodiment may include a cord coupled to the motor, the cord and the motor providing at least partial body weight support to a user during human gait training. In addition, the system of this embodiment may include a controller that actively adjusts the operation of the motor based upon measured gait parameters.

In one aspect of this embodiment, the controller adjusts the operation of the motor such that a force applied by the motor varies during a gait cycle.

In one aspect of this embodiment, the system may also include at least one gait-measuring device. In this aspect, the gait-measuring may be a device that measures body motion, limb-segment motion, joint motion, or a force or any combination thereof. In a particular embodiments of this aspect, the gait measuring device may be a load cell or a force plate.

In one aspect of this embodiment, the system may include a first gait-measuring device that provides a signal to the controller that indicates a position of the motor and a second gait-measuring device that provides a signal to the controller that indicates a force resulting from the gait of human.

In one aspect of this embodiment, the system may also include a frame. The frame of this aspect may be displaced over a treadmill or may be adapted to traverse a surface.

In one aspect of this embodiment, the system may also include a harness coupled to the cord and adapted to support a human.

In one embodiment, the present invention is directed to a method of human gait training. The method of this embodiment includes coupling a human to a motor; monitoring the gait of the human while the human is walking to produce a signal indicative of the gait of the human; and varying the operation of the motor based on the signal so as to assist the human in attaining a predetermined gait pattern.

In one aspect of this embodiment, coupling includes placing the human in a harness that is connected to the motor through at least a cord.

In one aspect of this embodiment, monitoring includes monitoring the motion of the center of mass of a system including the human.

In one aspect of this embodiment, monitoring includes measuring at least one of body motion, limb-segment motion, joint motion, or a force.

In one aspect of this embodiment, coupling includes coupling the human to a linear motor.

In one aspect of this embodiment, varying includes producing an error signal indicative of a current gait parameter of the human and a predetermined gait parameter.

### BRIEF DESCRIPTION OF THE DRAWINGS

The foregoing features of the invention will be more readily understood by reference to the following detailed description, taken with reference to the accompanying drawings, in which:

FIG. 1 is an example of one embodiment of a partial body weight support system according to the present invention;

FIG. 2 is a high-level block diagram of a control system according to one embodiment of the present invention;

FIG. 3 is a more detailed block diagram of one embodiment of a partial body weight support system shown in FIG. 2; and

FIG. 4 is a control diagram detailing the operation of one embodiment of a controller that may be utilized in an of partial body weight system according to the present invention.

### DETAILED DESCRIPTION OF SPECIFIC EMBODIMENTS

The limitations of the present technology in PBSW result in the need for additional therapist therapy to assist in gait training to assist in the appropriate loading of the limb throughout the stepping process. If the affected limbs were appropriately loaded, the action of, for example, a treadmill moving the feet would elicit appropriate reflex responses, and correct stepping could occur. However, current PBWS systems are incapable of modulating the level of support they provide. For example, during PBWS gait training using a passive spring, the patient is partially suspended over a treadmill, supported by an overhead frame and a spring tensioned to take a fraction of the person's weight. The spring suspension system provides variable support, but the support varies uncontrollably, providing the desired level of support only when the person passes through the position he/she was in when the spring tension was set. Much of the time the affected limb is either not in sufficient contact with the treadmill to be moved by it as desired, or is overloaded and, thus, collapses. Manual assistance is therefore required to maintain foot to treadmill contact, prevent collapse, and approximate normal joint ranges of movement.

Furthermore, when using a spring support, the vertical spring force in the current systems acting on the patient harness varies with the vertical position and is not constant. It does not reproduce the normal center of mass movement during gait. For patients, the phase of the stride at which the partial weight support is applied may be critical. The spring support has the effect of generating the highest support force in double limb support—when it is needed the least—and the lowest support force when the body is in single limb support —when it is needed the most.

Aspects of the present invention, therefore, may achieve one or more of the following results: 1) the partial weight support to produce the proper ground reaction forces and body center of mass movement; 2) proper phasing of the partial weight support; and 3) automatic operation for individual patients. In some embodiments, the system may be able to automatically adapt itself to an individual patient's needs, with the help of the physician and physical therapists, and then remember the PBWS gait training sequence that can be easily activated for the therapist for that session with that individual.

Furthermore, vertical oscillation of the center of mass during walking (from the highest point in single limb support to the lowest point in double limb support) is a key component to the energetics of walking, particularly in people with neurological or musculoskeletal injury, in whom the critical factor responsible for reducing center of mass oscillation (heel rise of the trailing limb during double limb support) is severely impaired. In addition, limited hip extension of the trailing limb in double limb support is an often-isolated specific impairment in a number of conditions. Embodiments of the present invention may take advantage of these discoveries by providing a system that may provide needed weight support, control the vertical oscillation of the body center of mass, or induce the full natural range of motion of the stepping limb or any combination of thereof.

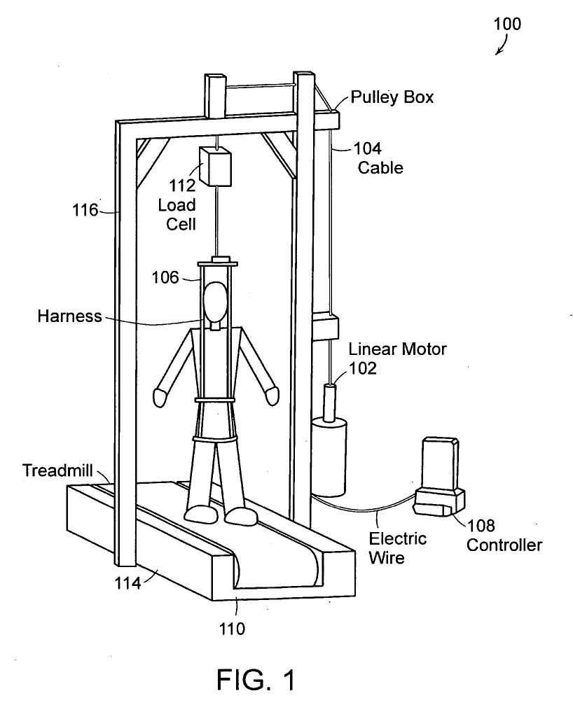

FIG. 1 is an example of one embodiment of a PBWS system according to the present invention. As shown, the system includes several elements. Of course, a system according to the present invention need not include each element shown in FIG. 1.

The system 100 of the embodiment of FIG. 1 includes a motor 102. The motor 102 may be coupled to a cable 104. In some embodiment, the cable 104 may be coupled to a harness 106 that is adapted to support a human. Of course, the harness is not required and could be omitted if other means for coupling the cable 104 to a human are present. For instance, the cable could be directly connected to an article of the human's clothing such as the human's belt (not shown). Regardless, the motor 102 and the cable 106 work together to, in some, embodiment, vary the amount of support provided to a human. In one embodiment, the motor 102 and the cable 106 work together to cause CoM trajectory of the human to rise during single limb stance and fall during double limb support, the natural pattern of gait.

The system of this embodiment also includes a controller 108. In general, the controller causes the motor to follow a predefined CoM trajectory. This may be accomplished by, for example, configuring the controller 108 to operate as a proportional-integral controller.

In one embodiment, the controller 108 may also receive a reference signal to which a current state of the system is compared and adjustments of location may be made. To that end, the current state of the system may be determined by including sensors that measure body motion, limb segment motion, joint motion, or a ground reaction force. Of course, other types of sensors could be used and, in some embodiments, multiple sensors may be used. For example, sensors that measure knee flexion angle or hip extension angle may be utilized.

The embodiment of FIG. 1 is provided with two different types of sensors. Specifically, the system shown in FIG. 1 includes a load cell 112 and force plates 110 which may, in some embodiments, be displaced within in treadmill 114. The force plates 110 could also be placed on any surface to be traversed. The system can be implemented with only one of either the load cell or a force plate. Of course, other sensors could be used and neither the load cell 112 nor the force plates 110 are required and are presented by way of example only.

By way of example, the load cell 112 may be employed to assist in force control of the system 100. In this example, a predefined percentage of the weight relief from the human is used, as a reference signal, and the load cell 112 measures produces a signal indicative of how much weight relief is being given. A comparison of these two signals may be provided to the controller 108 to adjust the operation of the motor. Similarly, the force plates 110 could also be utilized in this manor.

In some embodiments, the system 100 may also include a frame 116. The frame 116, in some embodiments, may support the cable 104, and consequently, a human. In one embodiment, the frame may be static and displaced above a treadmill 114. In other embodiments the frame 116 may be arranged and configured to allow it to traverse a surface, such as the ground or a floor. In such embodiments, the frame 116 may also include ground contacting members such as rollers or wheels (not shown). Of course, the specific type of frame need not be such as that shown in FIG. 1. For instance, the system 100 could be arranged such the internal supports of a building operated as the frame. In particular, the frame may be constructed of hollow aluminum beams and capable of supporting up to 350 lbs. In one embodiment, the frame 116 may be constructed of 0.076 m'0.15 m rectangular aluminum tubes. The dimensions of the frame may be 3.3 m in height to accommodate patients up to 1.8 m in height and 2 m in width to span the treadmill used to train the patient's gait.

The system may also include an optional safety circuit that allows a user of the system to monitor an emergency signal that protects a user from unexpected behavior of the motor or relieves the user from an uncomfortable situation. That is, the system may include, for example, an emergency shut down switch.

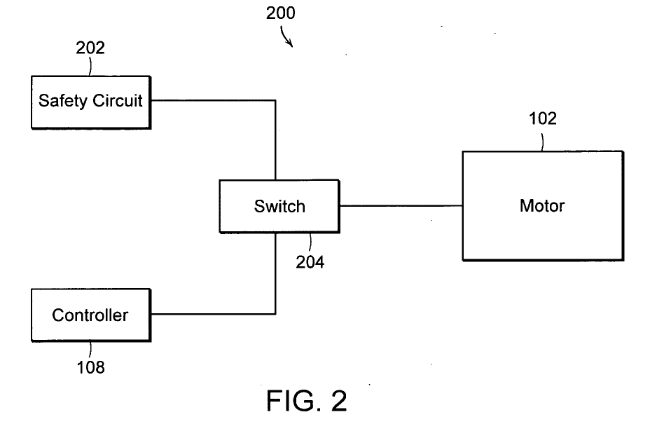

FIG. 2 is a high-level block diagram of a control system 200 according to one embodiment of the present invention. In this embodiment, the control system 200 includes an optional safety circuit 202 and a controller 108. The optional safety circuit 202 and the controller may both be configured to control the operation of the motor 102. To that end, an optional switch 204 may be employed to divert control of the motor from the controller 108 to the safety circuit 202 in the event of, for example, an emergency.

In one embodiment, the controller 108 may be an adaptive control systems that achieves, by controlling the motor 102, desired CoM kinematics appropriate for limb loading by utilizing position or force sensors, or both or other types of sensors, to determine the status of the system. That is, the controller 108 may utilize the output of sensors to determine whether the CoM trajectory rises during single limb stance and falls during double limb support, the natural pattern of gait. In some embodiments, CoM mean height and excursion will be adjusted to provide the needed unloading of the limbs, while maintaining adequate traction during stance. To meet this objective, the control system 108 may be arranged and configured to adaptively learn the time varying dynamics of the patient's gait, accommodating significant asymmetry in strength and functional control.

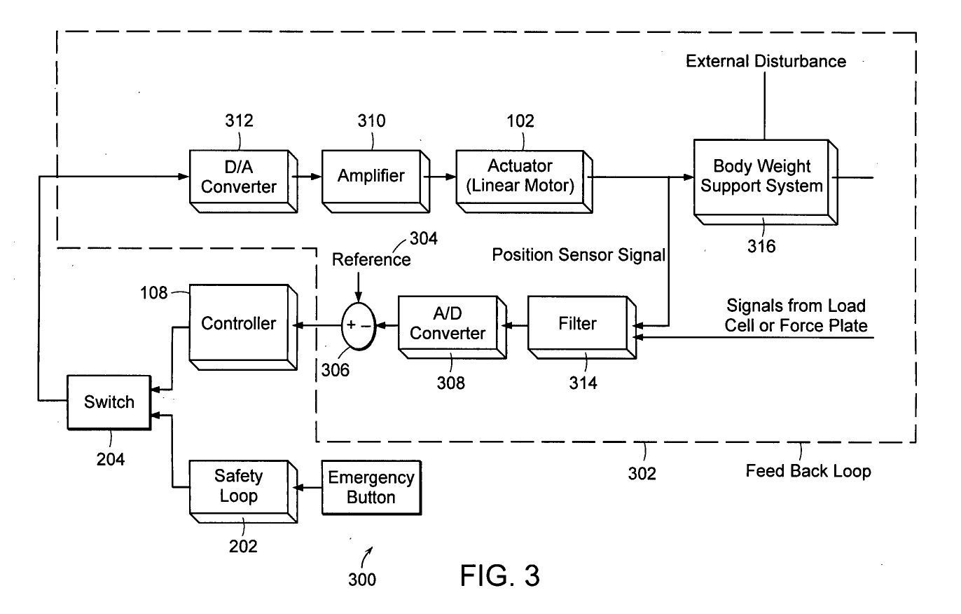

FIG. 3 shows the overall system block diagram of one embodiment of the present invention. The system shown in this embodiment includes an optional safety circuit 202, an optional switch 204, a controller 108 and a feedback loop 302. The feedback loop 302 serves to provide the controller 108 with information relating to the status of the system. For instance, the controller may receive information related to the position of the motor 102 as well as information received from one or more sensors. For example, the controller 108 may receive signals from the load cell or force plates described above.

Referring now more specifically to the feedback loop 302, a reference input 304 is included in the feedback loop 302. The reference input, in some embodiments, represents a normal person's dynamic CoM oscillating pattern. In some embodiments, the reference input 304 may be created by a signal generator (not shown). Of course, the reference input 304 may be created internal to the controller or in the same computer in which the controller is implemented. In one embodiment, the reference input 304 sets the desired motion pattern of the harness/patient and the desired amount of weight support. Based on the error between the reference input signals and the actual motion of the harness/patient (the output of summation block 306) the controller 108 determines a corrective action for the motor 102. A displacement sensor (not shown, and may be located within the motor 102) is used to measure the actual motion of the human (possibly by measuring the position of the motor shaft), and an amplifier 310 is used to drive the motor based 102 on output of the controller 108. In some embodiments, a load cell or force plate may be used to measure the actual harness forces as applied to the system during gait. As the output of the controller 108 is determined by a digital computer, the error signal, which is analog, is converted into a digital signal, by an A/D converter 308, before being fed into the controller. On the other hand, the output of the controller 308, which is digital, is converted into an analog signal, by a D/A converter 312, so as to drive the amplifier 310 and thereby control the motor 102. Of course, the information from the sensors in the system may need to be filtered, thus, the system of FIG. 3 may also include a filter 314 that smoothes or otherwise filters the sensor outputs. As shown in FIG. 3, force signals are the output of the body weight support system 316. The body weight support system 316, in some embodiments may include a human supported by a harness 106 coupled to the cable 104 (FIG. 1).

In one embodiment, the controller 108 may operate in such a manner that a human using a system according to the present invention exhibits a proper dynamic CoM oscillation displacement and a proper force pattern. A mathematic control algorithm producing the CoM motion/force trajectory may be created using a mathematical model of the system to be controlled. The algorithm may, in some embodiments, compute the appropriate corrective action so that the actual motion of the harness/patient and actual amount of weight support will quickly respond to changes in the input signals. In modern control theory, the controller design is carried out based on the mathematical model of the system to be controlled; here, the body weight support system comprised of the human being supported and that human's lower extremities. However, the lower extremity characteristics are different from patient to patient and often between limbs in an individual patient due to asymmetry. Thus, the lower extremity mathematical model may not be a perfect representation of each patient. Hence, the controller should be able to compensate for these inaccuracies; such a controller is referred to as a robust controller.

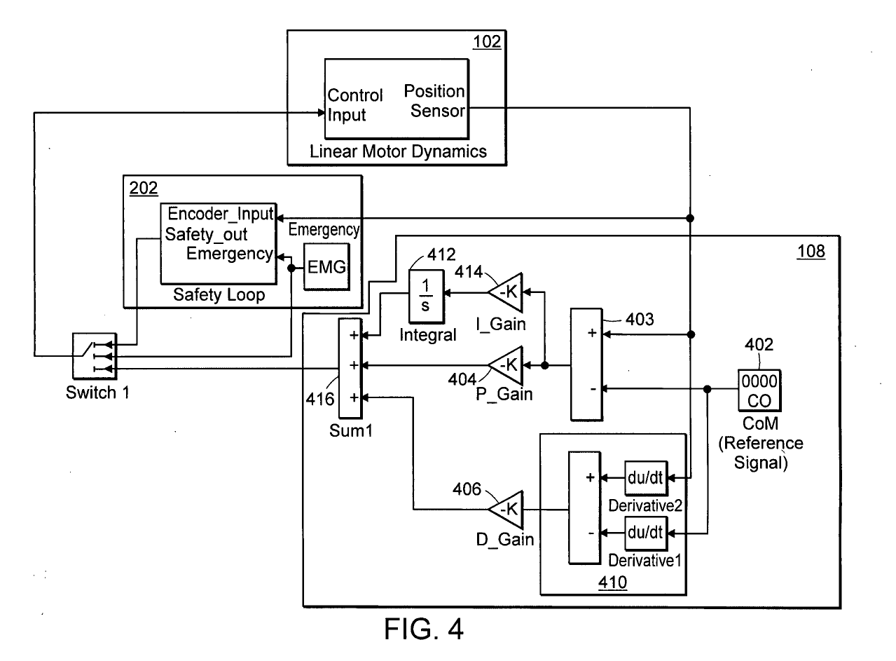

FIG. 4 shows an example of embodiment of a controller 104 that may meet some or all of the above constraints and be utilized in a partial body weight system according to the present invention.

In general, the controller 108 shown in FIG. 4 operates as proportional-integral (PI) position controller that has velocity feedback loop is used to make the shaft of the motor 102 vary such that a human using the system exhibits a predefined CoM trajectory. In one embodiment, the CoM trajectory may be sinusoidal. FIG. 4 also includes and optional safety circuit 202 that, in some embodiments, monitors an emergency signal and protects a patient from unexpected behavior of the motor or relieves a patient from an uncomfortable situation.

In the embodiment of the controller 108 shown in FIG. 4, the controller 108 includes a signal generator 402 that produces a reference signal which represents a normal person's dynamic center of mass oscillating pattern. Of course, the signal generator 402 could be external to the controller 108, as shown, for example, by the reference input 304 in FIG. 3. The reference signal is compared to the position as determined by the motor 104 by comparator 403. The feedback gains P_{Gain} 404 and D_{Gain} 406 are the active stiffness and a active damping relative to the desired position and velocity states. That is, the output of P_{Gain} 404 is related to the position of the shaft of the motor 102 and the output of D_{Gain} 406 is related to the velocity of the shaft of the motor 102. To that end, the position of the motor may be differentiated by standard derivative components such as differential block 410. These two gain terms increase the dynamic stiffness and damping by adding to the passive (mechanical) damping in the form

$$Dynamic Stiffness = \frac{{F}_{d}}{x} = m {s}^{2} + ( {c}_{m} + {D}_{Gain} ) s + {P}_{Gain}$$

Where F_{d} is the sum of the external disturbances. In addition to the command inputs for position and velocity, the controller 108 may also include an additional controller state: that of integrated position error ∫xₑᵣᵣₒᵣdt. The error value is the output of integrator 412. In addition, some gain may be required to operate on the error signal before it is integrated, thus, the system my also include an intergration gain 414 coupled between the output of the comparator 403 and the integrator 412. This system is further described in two articles entitled “Control System for Partial Body Weight Support Device for Human Gait Training,” copies of which are attached hereto and incorporated herein by reference.

In one embodiment, the command valued of the error state may always be zero, meaning no accumulated error is desired. This additional integrator assures that no steady-state position error is present if constant disturbances are present.

The sum of all the signals (or some other combination) may in some embodiments, be added in adder 416 and transferred to the motor 102 to control the operation thereof.

Although the above discussion discloses various exemplary embodiments of the invention, it should be apparent that those skilled in the art can make various modifications that will achieve some of the advantages of the invention without departing from the true scope of the invention.

What is claimed is:

1. A partial body-weight support system for human gait training comprising:
a motor;
a cord coupled to the motor, the cord and the motor providing at least partial body-weight support to a user during human gait training; and
a controller that actively adjusts the operation of the motor based upon measured gait parameters.

2. A system according to claim 1, wherein the controller adjusts the operation of the motor such that a force applied by the motor varies during a gait cycle.

3. A system according to claim 1, further comprising at least one gait-measuring device.

4. A system according to claim 3, wherein the at least one gait-measuring device is a device that measures body motion, limb segment motion, joint motion, or a force.

5. A system according to claim 4, wherein the gait measuring device is a load cell.

6. A system according to claim 4, wherein the gait measuring device is a force plate.

7. A system according to claim 3, wherein a first gait measuring device provides a signal to the controller that indicates a position of the motor and a second gait-measuring device provides a signal to the controller that indicates a force resulting from the gait of human.

8. A system according to claim 1, further comprising a frame.

9. A system according to claim 8, wherein the frame is displaced over a treadmill.

10. A system according to claim 8, wherein the frame is adapted to allow the frame to traverse a surface.

11. A system according to claim 1, further comprising a harness coupled to the cord and adapted to support a human.

12. A method of human gait training comprising:
coupling a human to a motor;
monitoring the gait of the human while the human is walking to produce a signal indicative of the gait of the human; and
varying the operation of the motor based on the signal so as to assist the human in attaining a predetermined gait pattern.

13. A method according to claim 12, wherein coupling includes placing the human in a harness that is connected to the motor through at least a cord.

14. A method according to claim 12, wherein monitoring includes monitoring the motion of the center of mass of a system including the human.

15. A method according to claim 12, wherein monitoring includes measuring at least one of body motion, limb-segment motion, joint motion, or a force.

16. A method according to claim 12, wherein coupling includes coupling the human to a linear motor.

17. A method according to claim 12, wherein the varying includes producing an error signal indicative of a current gait parameter of the human and a predetermined gait parameter.

18. A partial body-weight support system for training or diagnosis of a subject, the system utilizing a feedback controller that actively adjusts forces to or displacements of the subject's body based on measured gait parameters, wherein the magnitude of the forces to or displacements of the body varies throughout the subject's gait cycle.
